(19) United States
(12) Patent Application Publication
(10) Pub. No.: US 2025/0099749 A1
(43) Pub. Date: Mar. 27, 2025

(54) ELECTRICAL STIMULATION CUFF DEVICES AND SYSTEMS AND ELECTRODE ARRANGEMENTS THEREFOR

(71) Applicant: Boston Scientific Neuromodulation Corporation, Valencia, CA (US)

(72) Inventors: Michael A. Moffitt, Solon, OH (US); Rafael Carbunaru, Valley Village, CA (US); Ranjan Krishna Mukhari Nageri, Valencia, CA (US); Hari Hara Subramanian, Valencia, CA (US)

(21) Appl. No.: 18/886,646

(22) Filed: Sep. 16, 2024

Related U.S. Application Data
(60) Provisional application No. 63/539,774, filed on Sep. 21, 2023.

(51) Int. Cl. A61N 1/05; A61N 1/36

(52) U.S. Cl. CPC A61N 1/0556; A61N 1/36071; A61N 1/36185

(57) ABSTRACT

An electrical stimulation lead includes electrodes disposed on a cuff body and arranged into at least a first row, a second row, and a third row, where the second row is disposed between the first and third rows, and each electrode in the first row is electrically coupled to a corresponding electrode in the third row. In another electrical stimulation lead the electrodes are arranged so that the first and third rows each include multiple electrodes and the second row, between the first and third rows, includes at least one electrode with each electrode in the second row at least twice as long as each of the electrodes of the first and third rows. In another electrical stimulation lead the electrodes are arranged so that each electrode in the first and third rows is at least twice as long as each of the electrodes of the second row.

### CROSS-REFERENCE TO RELATED APPLICATIONS

This application claims the benefit under 35 U.S.C. § 119 (e) of U.S. Provisional Patent Application Ser. No. 63/539,774, filed Sep. 21, 2023, which is incorporated herein by reference.

### FIELD

The present disclosure is directed to the area of implantable electrical stimulation systems and methods of making and using the systems. The present disclosure is also directed to implantable electrical stimulation cuff devices, as well as methods of making and using the same.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients. Stimulation of the brain, such as deep brain stimulation, can be used to treat a variety of diseases or disorders.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

One aspect is an electrical stimulation lead that includes a cuff having a cuff body having an exterior surface and an interior surface and a plurality of electrodes disposed on the interior surface of the cuff body and arranged into at least three rows of electrodes, each of the rows including a plurality of the electrodes, wherein the at least three rows includes a first row, a second row, and a third row, wherein the second row is disposed between the first and third rows, wherein each electrode in the first row is electrically coupled to a corresponding electrode in the third row. The electrical stimulation lead also includes a lead body coupled to the cuff and a plurality of first conductors extending through the lead body and the cuff with the first conductors electrically coupled to the electrodes.

In at least some aspects, each electrode of the second row are staggered relative to the electrodes of the first and third rows. In at least some aspects, the first, second, and third rows have four electrodes each. In at least some aspects, the first, second, and third rows have six electrodes each. In at least some aspects, the first and third rows have n electrodes each and the second row has N-n electrodes, wherein N is a total number of electrodes and n is less than N.

In at least some aspects, the at least three rows further include a fourth row and a fifth row, wherein the first, second, and third rows are all disposed between fourth and fifth rows. In at least some aspects, the first and third rows have n electrodes each, the fourth and fifth rows have m electrodes each, and the second row has N-n-2m electrodes, wherein N is a total number of electrodes, n is less than N, m is less than N, and n-2m is less than N.

In at least some aspects, the cuff further includes a plurality of second conductors, wherein each of the second conductors electrically couples one of the electrodes of the first row to the corresponding one of the electrodes of the third row.

Another aspect is a method of electrically stimulating a patient. The method includes providing any of the electrical stimulation leads described above implanted within the patient and applying electrical current through the patient with at least one of the electrodes of each of the first and third rows having a first polarity and at least one of the electrodes of the second row having a second polarity opposite the first polarity.

A further aspect is an electrical stimulation lead that includes a cuff having a cuff body having an exterior surface and an interior surface and a plurality of electrodes disposed on the interior surface of the cuff body and arranged into at least three rows of electrodes, wherein the at least three rows includes a first row, a second row, and a third row, wherein the second row is disposed between the first and third rows, wherein the first and third rows each include a plurality of the electrodes and the second row includes at least one electrode, wherein each of the at least one electrode in the second row is at least twice as long as each of the electrodes of the first and third rows. The electrical stimulation lead also includes a lead body coupled to the cuff and a plurality of conductors extending through the lead body and the cuff with the conductors electrically coupled to the electrodes.

In at least some aspects, the at least three rows further includes a fourth row disposed between the first and third rows, wherein the fourth row includes at least one electrode, wherein each of the at least one electrode of the fourth row is at least twice as long as each of the electrodes of the first and third rows. In at least some aspects, the at least three rows further includes a fifth row that is not disposed between the first and third rows, wherein the fifth row includes at least one electrode, wherein each of the at least one electrode of the fifth row is at least twice as long as each of the electrodes of the first and third rows. In at least some aspects, the at least three rows further includes a fifth row that is between the second and fourth rows, wherein the fifth row includes at least one electrode.

In at least some aspects, the at least three rows further includes a fourth row that is not disposed between the first and third rows, wherein the fourth row includes at least one electrode, wherein each of the at least one electrode of the fourth row is at least twice as long as each of the electrodes of the first and third rows. In at least some aspects, each of the electrodes of the first row is electrically coupled to a corresponding one of the electrodes of the third row. In at least some aspects, the second row includes a plurality of the electrodes. In at least some aspects, the second row includes a single one of the electrodes that is at least as long as a combined length of all of the electrodes of the first row.

Another aspect is an electrical stimulation lead that includes a cuff having a cuff body having an exterior surface and an interior surface, and up to sixteen electrodes disposed on the interior surface of the cuff body and arranged into at least three rows of electrodes, wherein the at least three rows includes a first row, a second row, and a third row, wherein the second row is disposed between the first and third rows and includes more of the electrodes than the first row and more of the electrodes than the third row, wherein each electrode in the first and third rows is at least twice as long as each of the electrodes of the second row. The electrical stimulation lead also includes a lead body coupled to the cuff and a plurality of conductors extending through the lead body and the cuff with the conductors electrically coupled to the electrodes.

In at least some aspects, the first and third electrodes each include a plurality of the electrodes. In at least some aspects, the at least three rows further includes a fourth row that is not disposed between the first and third rows. In at least some aspects, the at least three rows further includes a fourth row that is disposed between the first and third rows. In at least some aspects, each of the electrodes of the first row is electrically coupled to a corresponding one of the electrodes of the third row.

Another aspect is a method of electrically stimulating a patient. The method includes providing any of the electrical stimulation leads described above implanted within the patient and applying electrical current through the patient using at least two of the electrodes of the cuff.

Yet another aspect is an electrical stimulation system that includes any of the electrical stimulation leads described above and a control module configured to receive a portion of the lead body of the electrical stimulation lead and to electrically couple to the electrodes of the cuff.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system that includes a lead electrically coupled to a control module;

FIG. 2A is a schematic view of one embodiment of the control module of FIG. 1 configured and arranged to electrically couple to an elongated device;

FIG. 2B is a schematic view of one embodiment of a lead extension configured and arranged to electrically couple the elongated device of FIG. 2A to the control module of FIG. 1;

FIG. 3A is a schematic view of one embodiment of an eight electrode arrangement for a cuff;

FIG. 3B is a schematic view of another embodiment of an eight electrode arrangement for a cuff;

FIG. 3C is a schematic perspective view of the electrode arrangement of FIG. 3A;

FIG. 3D is a schematic view of the cuff of FIG. 3A in a wrapped position;

FIGS. 4A to 4L are schematic views of additional embodiments of an eight electrode arrangement for a cuff;

FIGS. 5A to 5I are schematic views of various embodiments of a twelve electrode arrangement for a cuff;

FIGS. 6A and 6B are schematic views of two embodiments of an eight channel electrode arrangement for a cuff with two rows of electrodes that are electrically coupled in opposing pairs of electrodes;

FIGS. 6C and 6D are schematic views of two embodiments of a twelve channel electrode arrangement for a cuff with two rows of electrodes that are electrically coupled in opposing pairs of electrodes;

FIGS. 7A to 7D are schematic views of additional embodiments of electrode arrangements for a cuff with two rows of electrodes that are electrically coupled in opposing pairs of electrodes;

FIGS. 8A to 8E are schematic views of further embodiments of electrode arrangements for a cuff with two rows of electrodes that are electrically coupled in opposing pairs of electrodes; and

FIG. 9 is a schematic block diagram of one embodiment of an electrical stimulation arrangement.

### DETAILED DESCRIPTION

The present invention is directed to the area of implantable electrical stimulation systems and methods of making and using the systems. The present invention is also directed to implantable electrical stimulation cuff devices, as well as methods of making and using the same.

Suitable implantable electrical stimulation systems include, but are not limited to, a least one lead with one or more electrodes disposed along a distal end of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,203,548; 7,244,150; 7,450,997; 7,596,414; 7,610,103; 7,672,734;7,761,165; 7,783,359; 7,792,590; 7,809,446; 7,949,395; 7,974,706; 6,175,710; 6,224,450; 6,271,094; 6,295,944; 6,364,278; 6,391,985; 7,596,414; 7,974,706; 8,831,742; 8,423,157; 10,485,969; 10,493,269; 10,709,888 and 10,485,969; and U.S. Patent Applications Publication Nos. 2007/0150036; 2008/0304830; 2009/0187222; 2009/0276021; 2010/0076535; 2010/0268298; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; 2017/0333692; 2018/0154156; 2022/0370793; and 2022/0395690, all of which are incorporated by reference in their entireties.

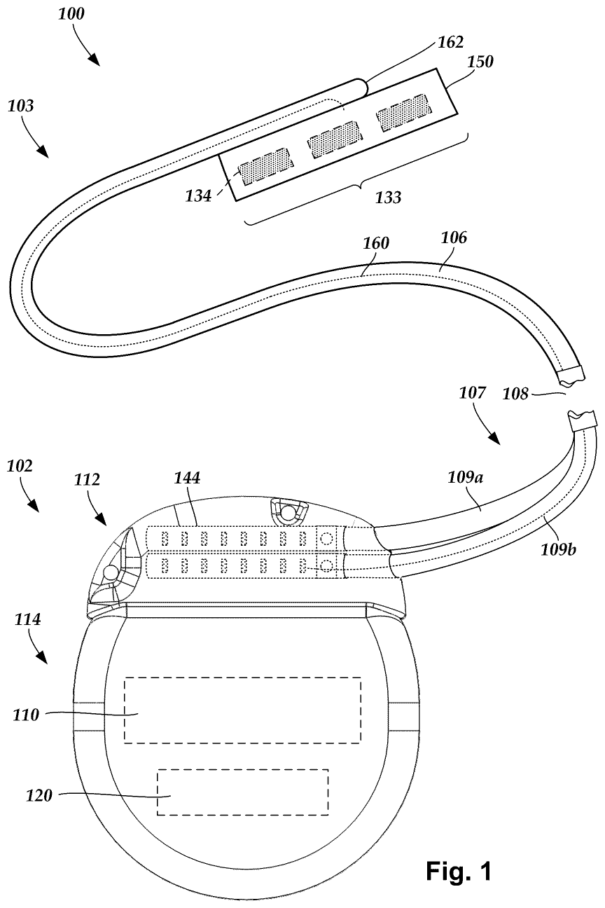

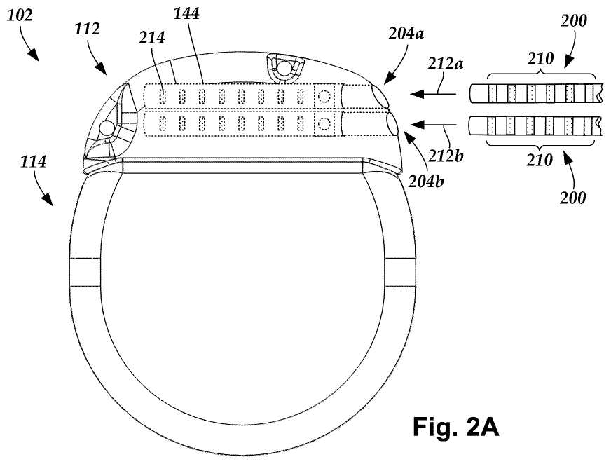

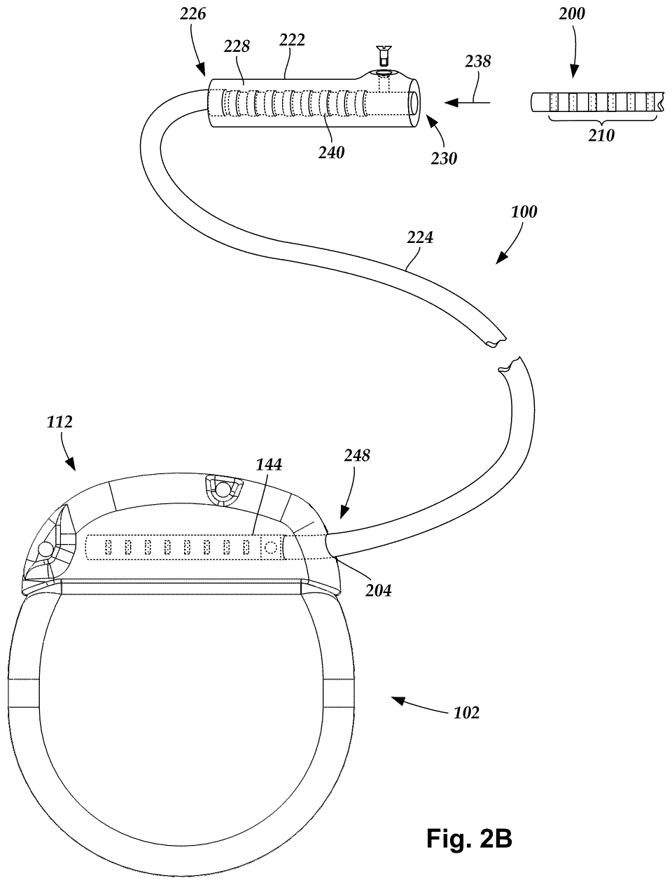

FIG. 1 illustrates schematically one embodiment of an electrical stimulation system 100. The electrical stimulation system includes a control module (e.g., a stimulator or pulse generator) 102 and a lead 103 coupleable to the control module 102. The lead 103 includes a mount 162 and a cuff 150. The lead 103 includes one or more lead bodies 106, an array 133 of electrodes, such as electrode 134, and an array of terminals (e.g., 210 in FIG. 2A-2B) disposed within the cuff 150 attached to the one or more lead bodies 106. In at least some embodiments, the lead is isodiametric along at least a portion of the longitudinal length of the lead body 106. FIG. 1 illustrates one lead 103 coupled to a control module 102. Other embodiments may include two, three, four, or more leads 103 coupled to the control module 102. In yet other embodiments, a lead 103 may be coupled to multiple control modules 102. For example, a lead with 64 electrodes may be coupled to two control modules 102 that are capable of handling 32 electrodes each.

The lead 103 can be coupled to the control module 102 in any suitable manner. In at least some embodiments, the lead 103 couples directly to the control module 102. In at least some other embodiments, the lead 103 couples to the control module 102 via one or more intermediate devices (200 in FIGS. 2A-2B). For example, in at least some embodiments one or more lead extensions 224 (see e.g., FIG. 2B) can be disposed between the lead 103 and the control module 102 to extend the distance between the lead 103 and the control module 102. Other intermediate devices may be used in addition to, or in lieu of, one or more lead extensions including, for example, a splitter, an adaptor, a device with multiplexing capability (as described, for example, in U.S. Pat. No. 10,350,413, which is incorporated by reference in its entirety) or the like or combinations thereof. It will be understood that, in the case where the electrical stimulation system 100 includes multiple elongated devices disposed between the lead 103 and the control module 102, the intermediate devices may be configured into any suitable arrangement.

In FIG. 1, the electrical stimulation system 100 is shown having a splitter 107 configured and arranged for facilitating coupling of the lead 103 to the control module 102. The splitter 107 includes a splitter connector 108 configured to couple to a proximal end of the lead 103, and one or more proximal tails 109a and 109b configured and arranged to couple to the control module 102 (or another splitter, a lead extension, an adaptor, or the like). The splitter 107 and splitter connector 108 may be part of the lead 103 or may be a separate component that attaches to the lead.

The control module 102 typically includes a connector housing 112 and a sealed electronics housing 114. Stimulation circuitry 110 and an optional power source 120 are disposed in the electronics housing 114. A control module connector 144 is disposed in the connector housing 112. The control module connector 144 is configured and arranged to make an electrical connection between the lead 103 and the stimulation circuitry 110 of the control module 102.

The electrical stimulation system or components of the electrical stimulation system, including the lead body 106 and the control module 102, are typically implanted into the body of a patient. The electrical stimulation system can be used for a variety of applications including, but not limited to, brain stimulation, neural stimulation, spinal cord stimulation, muscle stimulation, and the like.

The lead body 106 can be made of, for example, a non-conductive, biocompatible material such as, for example, silicone, polyurethane, polyetheretherketone (“PEEK”), epoxy, and the like or combinations thereof. The lead body 106 may be formed in the desired shape by any process including, for example, molding (including injection molding), casting, and the like. The non-conductive material typically extends from the distal end of the lead body 106 to the proximal end of the lead body 106.

Terminals (e.g., 210 in FIGS. 2A-2B) are typically disposed along the proximal end of the lead body 106 of the electrical stimulation system 100 (as well as any splitters, lead extensions, adaptors, or the like) for electrical connection to corresponding connector contacts (e.g., 214 and 240 in FIG. 2B). The connector contacts are disposed in connectors (e.g., 144 in FIGS. 1-2B; and 222 in FIG. 2B) which, in turn, are disposed on, for example, the control module 102 (or a lead extension, a splitter, an adaptor, or the like). Electrically conductive wires 160, cables, or the like (only one of which is shown in FIG. 1) extend from the terminals to the electrodes 134. Typically, one or more electrodes 134 are electrically coupled to each terminal. In at least some embodiments, each terminal is only connected to one electrode 134.

The electrically conductive wires (“conductors”) 160 (only one of which is illustrated in FIG. 1 for clarity) may be embedded in the non-conductive material of the lead body 106 or can be disposed in one or more lumens (not shown) extending along the lead body 106. In some embodiments, there is an individual lumen for each conductor. In other embodiments, two or more conductors extend through a lumen. There may also be one or more lumens (not shown) that open at, or near, the proximal end of the lead body 106, for example, for inserting a stylet to facilitate placement of the lead body 106 within a body of a patient. Additionally, there may be one or more lumens (not shown) that open at, or near, the distal end of the lead body 106, for example, for infusion of drugs or medication into the site of implantation of the lead body 106. In at least one embodiment, the one or more lumens are flushed continually, or on a regular basis, with saline, epidural fluid, or the like. In at least some embodiments, the one or more lumens are permanently or removably sealable at the distal end.

FIG. 1 also illustrates a mount 162, part of the lead body 106, coupled to cuff 150. The conductors 160 (only one of which is illustrated in FIG. 1 for clarity) from within the lead body 106 are received in the mount 162, which in turn is attached to the cuff 150 such that each conductor passes through the mount 162 for a direct electrical connection with one of the electrodes 134 (e.g., one conductor is electrically connected with one electrode and so on). The mount 162 may be attached using a variety of means such as, but not limited to, molding or adhering the mount 162 to the cuff 150. In other embodiments, the conductors 160 from within the lead body 106 are electrically coupled to the electrodes 134 using jumper, intermediate or transition wires from the lead body 106 to the electrodes 134.

The mount 162 can be offset from the cuff 150, as illustrated in FIG. 1, or in-line with the cuff or in any other suitable arrangement. Examples of cuff leads 103 can be found at U.S. Pat. Nos. 7,596,414; 7,974,706; 8,423,157; 10,485,969; 10,493,269; 10,709,888; and 10,814,127; and U.S. Patent Application Publications Nos. 2017/0333692; 2018/0154156; 2022/0370793; and 2022/0395690, all of which are incorporated herein by reference in their entireties.

FIG. 2A is a schematic side view of one embodiment of a proximal end of one or more elongated devices 200 configured and arranged for coupling to one embodiment of the control module connector 144. The one or more elongated devices may include, for example, the lead body 106, one or more intermediate devices (e.g., the lead extension 224 of FIG. 2B, an adaptor, or the like or combinations thereof), or a combination thereof. FIG. 2A illustrates two elongated devices 200 coupled to the control module 102. These two elongated devices 200 can be two tails as illustrated in FIG. 1 or two different leads or any other combination of elongated devices.

The control module connector 144 defines at least one port into which a proximal end of the elongated device 200 can be inserted, as shown by directional arrow 212. In FIG. 2A (and in other figures), the connector housing 112 is shown having two ports 204a and 204b. The connector housing 112 can define any suitable number of ports including, for example, one, two, three, four, five, six, seven, eight, or more ports.

The control module connector 144 also includes a plurality of connector contacts, such as connector contact 214, disposed within each port 204a and 204b. When the elongated device 200 is inserted into the ports 204a and 204b, the connector contacts 214 can be aligned with a plurality of terminals 210 disposed along the proximal end(s) of the elongated device(s) 200 to electrically couple the control module 102 to the electrodes (134 of FIG. 1) disposed at a distal end of the lead 103. Examples of connectors in control modules are found in, for example, U.S. Pat. Nos. 7,244,150 and 8,224,450, which are incorporated by reference in their entireties.

FIG. 2B is a schematic side view of another embodiment of the electrical stimulation system 100. The electrical stimulation system 100 includes a lead extension 224 that is configured and arranged to couple one or more elongated devices 200 (e.g., the lead body 106, an adaptor, another lead extension, or the like or combinations thereof) to the control module 102. In FIG. 2B, the lead extension 224 is shown coupled to a single port 204 defined in the control module connector 144. Additionally, the lead extension 224 is shown configured and arranged to couple to a single elongated device 200. In alternate embodiments, the lead extension 224 is configured and arranged to couple to multiple ports 204 defined in the control module connector 144, or to receive multiple elongated devices 200, or both.

A lead extension connector 222 is disposed on the lead extension 224. In FIG. 2B, the lead extension connector 222 is shown disposed at a distal end 226 of the lead extension 224. The lead extension connector 222 includes a connector housing 228. The connector housing 228 defines at least one port 230 into which terminals 210 of the elongated device 200 can be inserted, as shown by directional arrow 238. The connector housing 228 also includes a plurality of connector contacts, such as connector contact 240. When the elongated device 200 is inserted into the port 230, the connector contacts 240 disposed in the connector housing 228 can be aligned with the terminals 210 of the elongated device 200 to electrically couple the lead extension 224 to the electrodes (134 of FIG. 1) disposed along the lead (103 in FIG. 1).

In at least some embodiments, the proximal end of the lead extension 224 is similarly configured and arranged as a proximal end of the lead 103 (or other elongated device 200). The lead extension 224 may include a plurality of electrically conductive wires (not shown) that electrically couple the connector contacts 240 to a proximal end 248 of the lead extension 224 that is opposite to the distal end 226. In at least some embodiments, the conductive wires disposed in the lead extension 224 can be electrically coupled to a plurality of terminals (not shown) disposed along the proximal end 248 of the lead extension 224. In at least some embodiments, the proximal end 248 of the lead extension 224 is configured and arranged for insertion into a connector disposed in another lead extension (or another intermediate device). In other embodiments (and as shown in FIG. 2B), the proximal end 248 of the lead extension 224 is configured and arranged for insertion into the control module connector 144.

The arrangement of electrodes on a cuff can be used for selectivity of the portions of the nerve that are stimulated. Many conventional control modules support eight or twelve electrodes. It is desirable to arrange these electrodes in patterns that provide stimulation selectivity.

Many conventional cuffs include electrodes that surround, or nearly or substantially surround, around the nerve when the cuff is implanted around the nerve. For vagus nerve stimulation, as an example, such electrode systems often stimulate a large section of the fibers on one side of the vagus nerve. Stimulation of the axons on the other side of the vagus nerve can require higher amplitudes, which may increase the likelihood of causing side-effects, such as hoarseness, cough, dysarthria, or the like. Arranging electrodes to provide radial selectivity can improve therapy by stimulating therapy-related axons while avoiding or reducing stimulation of side-effect-related axons as compared to the conventional cuffs described above.

In at least some embodiments, cuffs with an electrode arrangement that includes at least 3 circumferential rows of electrodes with the rows disposed at different longitudinal positions along the nerve axis can facilitate fiber-type selectivity to improve therapy. Such electrode arrangements can provide circumferential selectivity, longitudinal selectivity, or both. In many instances, fibers associated with side-effects are larger than those associated with therapeutic effects.

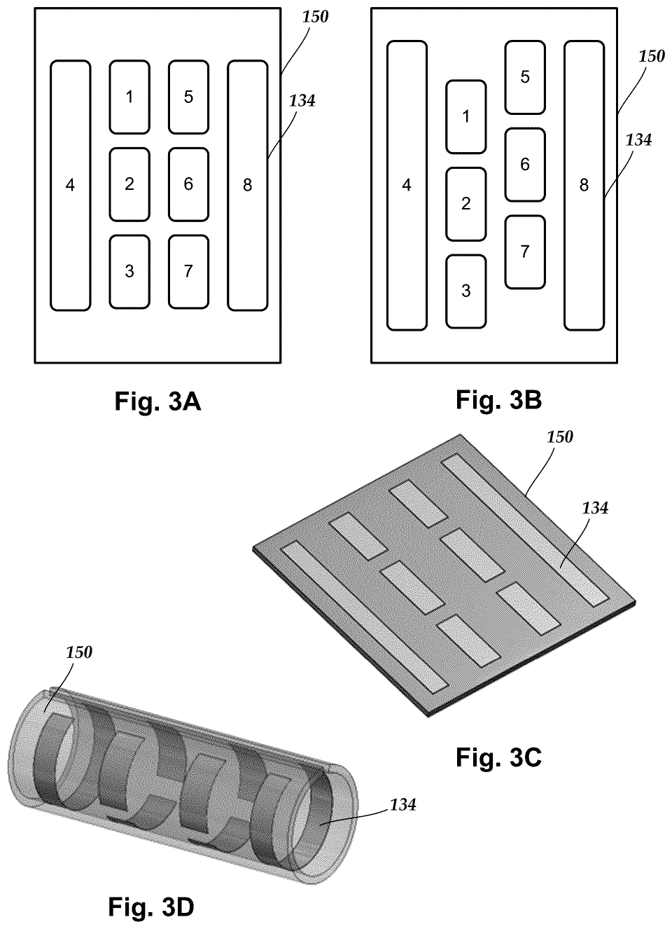

FIGS. 3A to 3D illustrate embodiments of the cuff 150 of a cuff lead. FIGS. 3A and 3B are plan views of two embodiments of the cuff 150 unwrapped to illustrate the arrangement of the electrodes 134. FIG. 3C illustrates the embodiment of FIG. 3A in a perspective view and FIG. 3D illustrates that embodiment wrapped into a cuff as when it would be disposed around a nerve or other tissue. It is noted that, as illustrated in FIG. 3D, the cuffs 150 illustrated in FIGS. 3A and 3B are to be wrapped around a horizontal axis. In the embodiments of FIGS. 3A and 3B, the vertical edges become the circumferential edges when the cuff is wrapped, as illustrated in FIG. 3D.

In the cuffs 150 of FIGS. 3A and 3B, the arrangement of the electrodes 134, there are two longer electrodes, labeled “4” and “8”, and two rows of three electrodes each, labeled “1”, “2”, “3” and “4”, “5”, “6”, respectively. The four rows provide four different longitudinal positions for stimulation. In FIG. 3A, the two rows of three electrodes provide three different circumferential stimulation positions. In the arrangement of FIG. 3B, one row is staggered relative to the other row. In the staggered embodiment of FIG. 3B, there are six different circumferential stimulation positions. It will be understood that for any of the embodiments illustrated or described herein, one or more of the rows can be staggered relative to another of the rows.

In at least some embodiments, the length of the longer electrodes is at least two, three, or more times the length of the shorter electrodes. In some embodiments, adjacent rows of electrodes are separated by the same distance, for example, at least 0.2, 0.5, 1, 2, 3, 4, 5, 6, 7, 8, 9, or 10 mm (or any other suitable distance).

In other embodiments, the separate distance between different pairs of adjacent rows of electrodes can be different. For example, the separation distance between the second and third rows of electrodes (i.e., the two rows with three electrodes) can be less than the separation distance between the first/second rows and third/fourth rows. In at least some embodiments, electrodes that are relatively close to each other can be used for anode-cathode pairs that are very narrow. In at least some embodiments, the separation distance between the two rows with three electrodes is no more than 5, 4, 3, 2, 1, or 0.5 mm. In at least some embodiments, the separation distance between the first/second rows and the third/fourth rows is larger and is at least 3, 5, 8, or 10 mm. In at least some embodiments, the separation distance between the first/second rows and the third/fourth rows is at least twice, three times, or four times the separation distance between the second/third rows.

As illustrated in FIG. 3D, when the cuff is wrapped around the nerve or other tissue, the two longer electrodes, labeled “4” and “8”, form a ring or most of a ring around the nerve or other tissue. The two rows of three electrodes, labeled “1”, “2”, “3” and “4”, “5”, “6”, respectively, allow for radial selectivity, for example, when one or more of the smaller electrodes are selected as the active electrode(s) and one of the two longer electrodes (or any of the smaller electrodes) are selected as the return electrode(s). In at least some embodiments, the longer electrodes can provide a convenient return electrode relative to one or more of the shorter electrodes acting as an active electrode.

In FIG. 3A, the arrangement of electrodes provide three different radial stimulation sites in order to stimulate the different fibers or other portions of the nerve that the cuff is wrapped around. In FIG. 3B, the arrangement of electrodes provide six different radial stimulation sites due to the staggered electrode arrangement. It will be understood that utilizing multiple electrodes, with the same or different stimulation amplitude, can provide stimulation centers ranging along the row.

In at least some embodiments, selecting a cathode and an anode in rows adjacent to each other can better stimulate smaller, closer fibers. To enhance selectivity, anode (or cathode) intensification techniques can be used. In at least some embodiments, anode intensification can be achieved by assigning a substantial portion (for example, at least 10, 15, 25, 30, 33, 35, or 40 percent) of the cathodic current to the housing or case of the control module so that the anode(s) appear to be relatively stronger than the cathode(s). As an example, 20% of the anodic current can be assigned to the single electrode in the first row, 60% of the cathodic current can be assigned to one of the electrodes in the second row, 80% of the anodic current can be assigned to the corresponding electrode in the third row, 5% of the cathodic current can be assigned to the single electrode in the fourth column, and 35% of the cathodic current is assigned to the housing of the control module.

In at least some embodiments, anode intensification is used for tripolar stimulation. In at least some embodiments, one of the ring electrodes (i.e., the single electrode of the first or fourth row) can be used to reduce the leakage of current outside of the cuff by creating a sink or source with opposite polarity to the nearest active electrode. Anode intensification can be used for leakage reduction.

In at least some embodiments, unidirectional propagating action potentials can be generated using one or more cathodes and a strong anode can block action potentials in the non-propagating direction. The anode can be, for example, one or more of the electrodes of the first, third, or fourth row with the cathodic current assigned to one or more of the electrodes of the second row. In at least some embodiments, the anode is strengthened by assigning a substantial portion (for example, at least 10, 15, 25, 30, 33, 35, or 40 percent) of the cathodic current to the housing of the control module.

It will be understood that cathode intensification can be achieved by reversing the polarities described above.

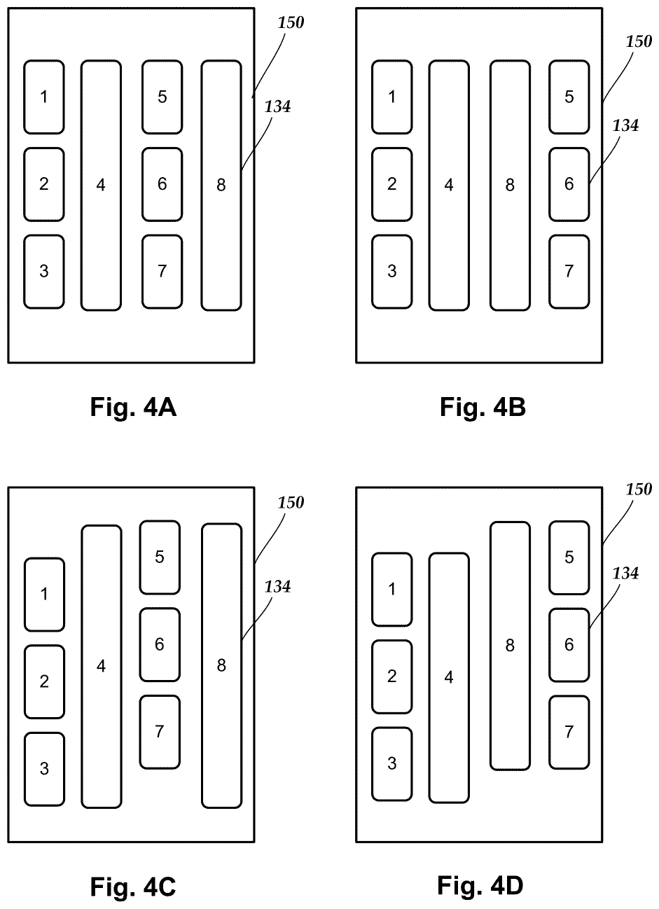

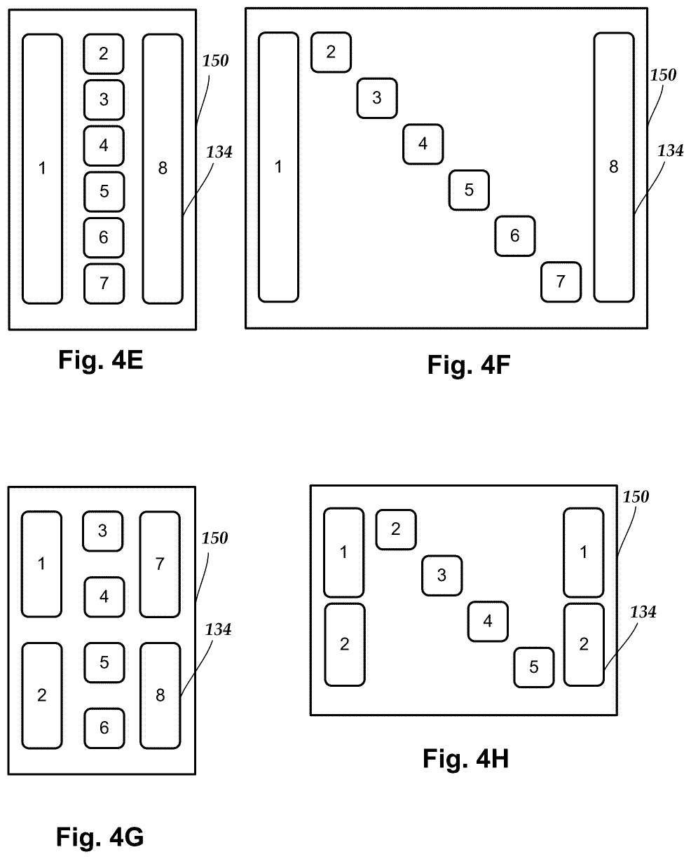

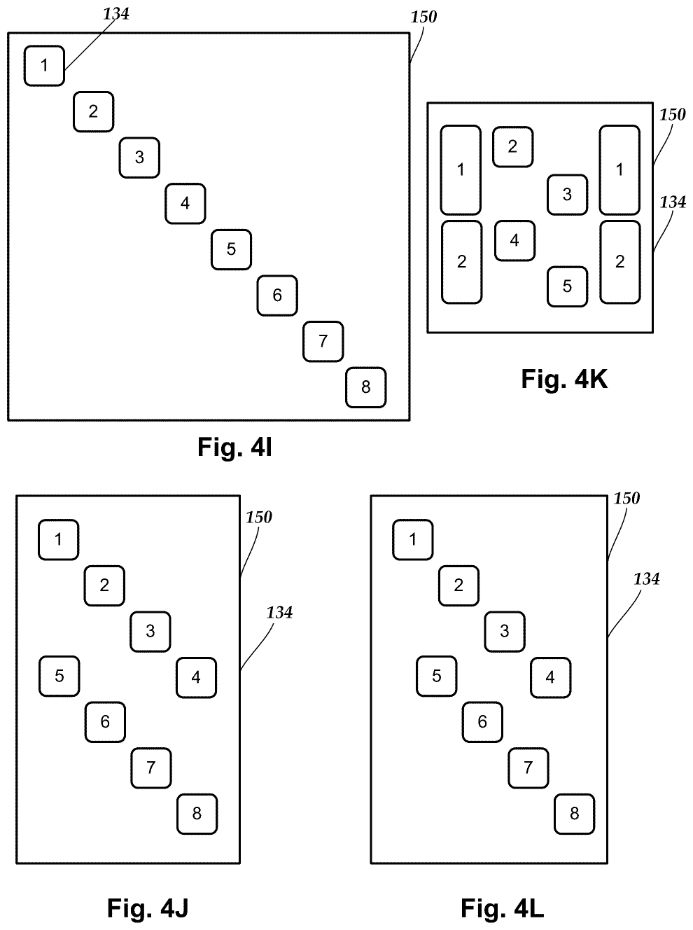

The arrangements in FIGS. 3A and 3B can be referred to as 1-3-3-1 arrangements where each number represents the number of electrodes in the individual rows. FIGS. 4A to 4D illustrated additional embodiments of a cuff 150 with four rows of electrodes that are rearranged relative to the embodiments of FIGS. 3A and 3B. FIGS. 4A and 4C illustrate 3-1-3-1 electrode arrangements with FIG. 4C illustrating one row of three electrodes staggered relative to the other. It will be understood that, for any of the electrode arrangements presented herein, the inverted arrangement (e.g., 1-3-1-3 is the inverted arrangement of 3-1-3-1) or any other order of the rows can also be used. FIGS. 4B and 4D illustrate 3-1-1-3 electrode arrangements with FIG. 4D illustrating one row of three electrodes staggered or offset relative to the other. FIG. 4E illustrates a 1-6-1 arrangement. FIG. 4G illustrates a 2-4-2 arrangement.

FIGS. 4F, 4H, 4I, 4J, 4K, and 4L illustrate arrangements with arrangements of electrodes that form one or two helices when the cuff is wrapped around. FIG. 4I illustrates a single helix of eight electrodes which provides eight longitudinal positions and eight circumferential positions. In at least some embodiments, more longitudinal positions can improve selectivity. Moreover, in at least some instances, the axons can move within the nerve. In at least some embodiments, more longitudinal positions can facilitate additional multipole configurations with different lengths.

FIG. 4J illustrates two helices of four electrodes each which provides four longitudinal positions and seven (or eight if electrodes “4” and “5” are not aligned) circumferential positions. If the electrodes of one helix are instead offset longitudinally from the electrodes of the other helix, as illustrated, for example, in FIG. 4L, then the number of longitudinal positions can be increased relative to the embodiment illustrated in FIG. 4J and denser than the longitudinal positions of the embodiment illustrated in FIG. 4I.

FIG. 4F illustrates a single helix of six electrodes flanked by two longer electrodes. In at least some embodiments, the longer electrodes can source or sink current from within the cuff to prevent or reduce current leakage. Another arrangement can include two helices of three electrodes each with the helices flanked by two longer electrodes. In at least some embodiments, the two longer electrodes have a length equal to, or larger than, the length of the electrodes of the helix in the circumferential direction (i.e., the vertical direction in the Figures). FIG. 4H illustrates a single helix of four electrodes flanked by two rows of two electrodes each. In at least some embodiments, the flanking electrodes are closer in size to the other electrodes to better orient the stimulation field. FIG. 4K illustrates a two helices of two electrodes each and flanked by two rows of two electrodes each. In at least some embodiments, the flanking electrodes can have a length in the circumferential direction that is equal to, or longer than, the length in the circumferential direction of one, two, three, four, or more of the electrodes in the helix or helices.

In at least some embodiments, the longitudinal and circumferential separations between adjacent electrodes can be uniform, as shown in the illustrated embodiments. In at least some embodiments, the longitudinal and circumferential separations between adjacent electrodes can be different for different pairs of adjacent electrodes. In at least some embodiments, different distances can provide flexibility in the selection of bipolar length or multipole arrangement.

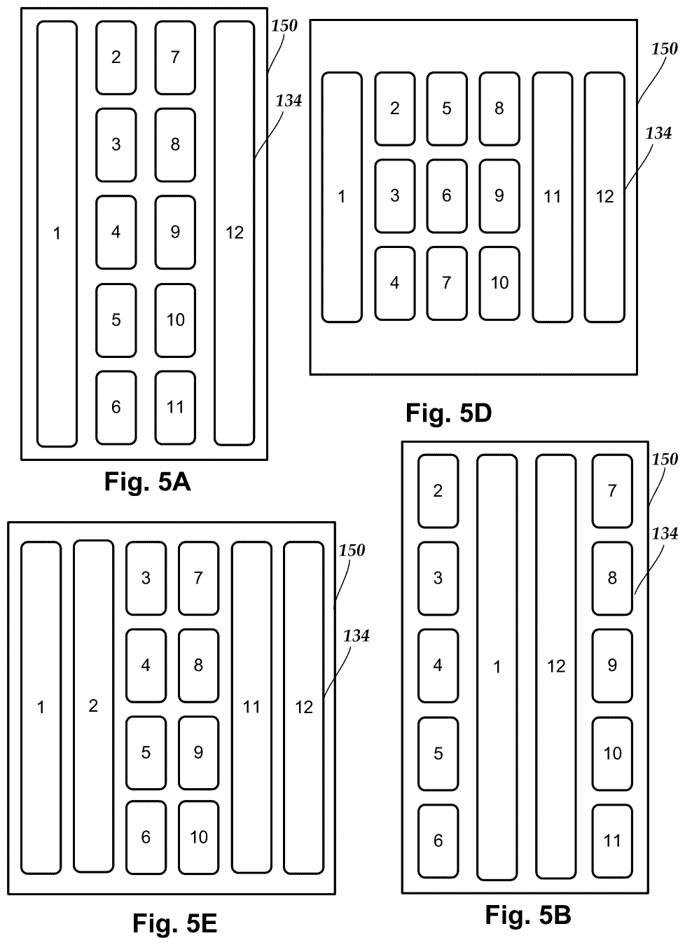

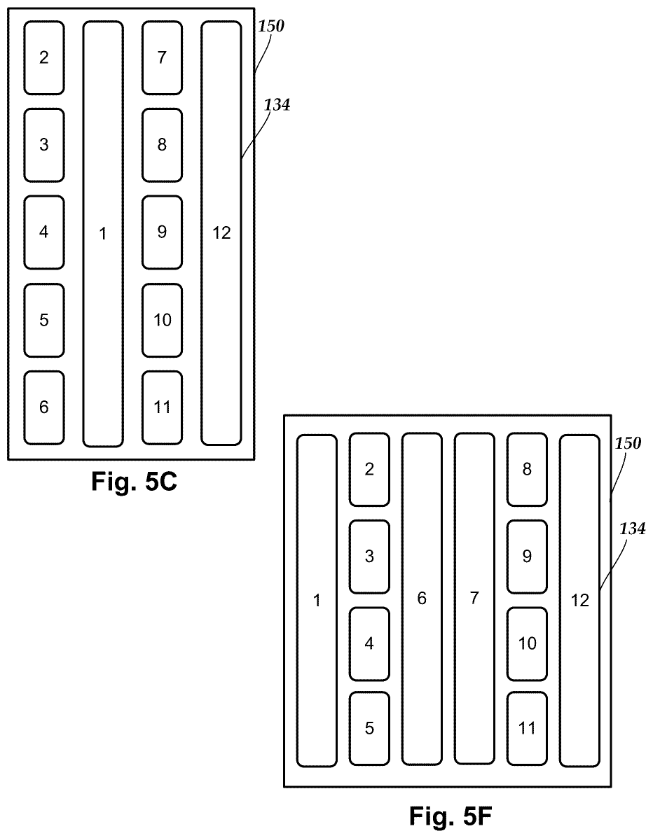

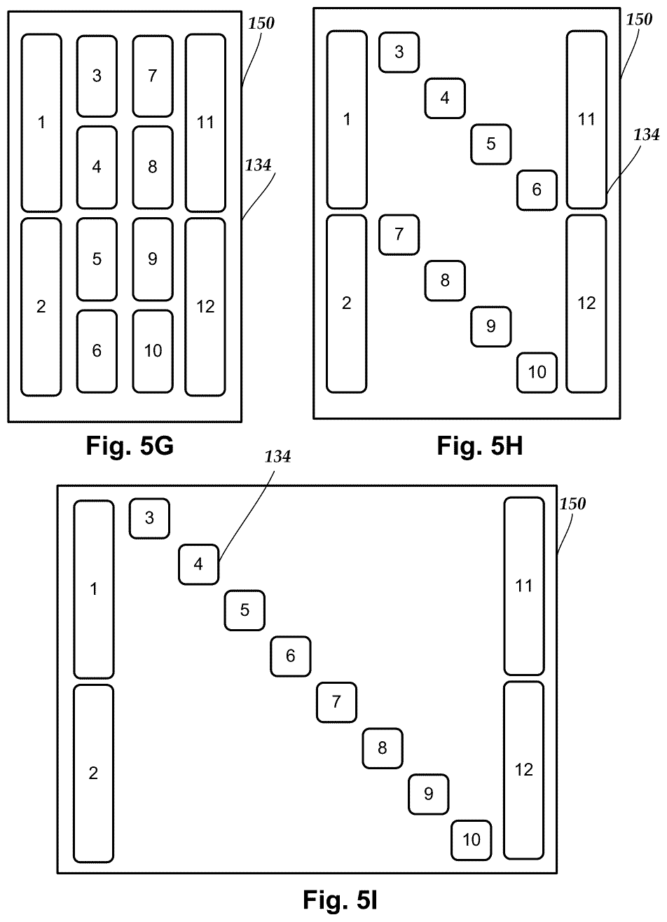

FIGS. 5A to 5I illustrate similar embodiments to some of those illustrated in FIGS. 4A to 4L except these embodiments have twelve electrodes instead of eight electrodes. The same considerations and variations presented above for the eight electrode embodiments can be applied to these twelve electrode embodiments. FIG. 5A, 5B, and 5C illustrate 1-5-5-1, 5-1-1-5, and 5-1-5-1 arrangements, respectively. Any other arrangements of two rows of five electrodes each and two rows of one electrode each can be used (e.g., 1-5-1-5). FIGS. 5D, 5E, and 5F illustrate 1-3-3-3-1-1, 1-1-4-4-1-1, and 1-4-1-1-4-1 arrangements. In at least some embodiments, these arrangement provide for multiple bipolar lengths or multipolar arrangements. In at least some embodiments, the 1-4-1-1-4-1 arrangement can provide two distinct longitudinal positions with circumferential selectivity. Any other arrangements of three rows of three electrodes each and three rows of one electrode each can be used (e.g., 1-3-1-3-1-3, 1-3-1-1-3-3, or 1-3-3-1-1-3). Any other arrangements of two rows of four electrodes each and four rows of one electrode each can be used (e.g., 1-4-1-4-1-1). FIG. 5G illustrates a 2-4-4-2 arrangement. Any other arrangements of two rows of four electrodes each and two rows of two electrodes each can be used (e.g., 2-4-2-4 or 4-2-2-4). FIGS. 5H and 5I illustrate embodiments with two and one helices, respectively. Any of the other arrangements that include one or two helices, such as those illustrated in FIGS. 4F, 4H, 4I, 4J, and 4K, can be adapted to twelve electrodes.

Electrode arrangements similar to those presented in FIGS. 4A to 4L and 5A to 5I for any selected number of electrodes can be provided. For example, for N electrodes, the following electrode arrangements can be provided ((N/2-1)/2)-1-((N/2-1)/2)-1, ((N/2-1)/2)-1-1-((N/2-1)/2), 1-((N/2-1)/2)-((N/2-1)/2)-1, 1-(N-2)-1, 2-(N-4)-2, 1-((N-1)/2)-((N-1)/2)-1, 1-1-((N-2)/2)-((N-2)/2)-1-1, or the like or any other arrangement of these rows. In addition, the arrangements with one or two helices can be adapted to any selected number of electrodes.

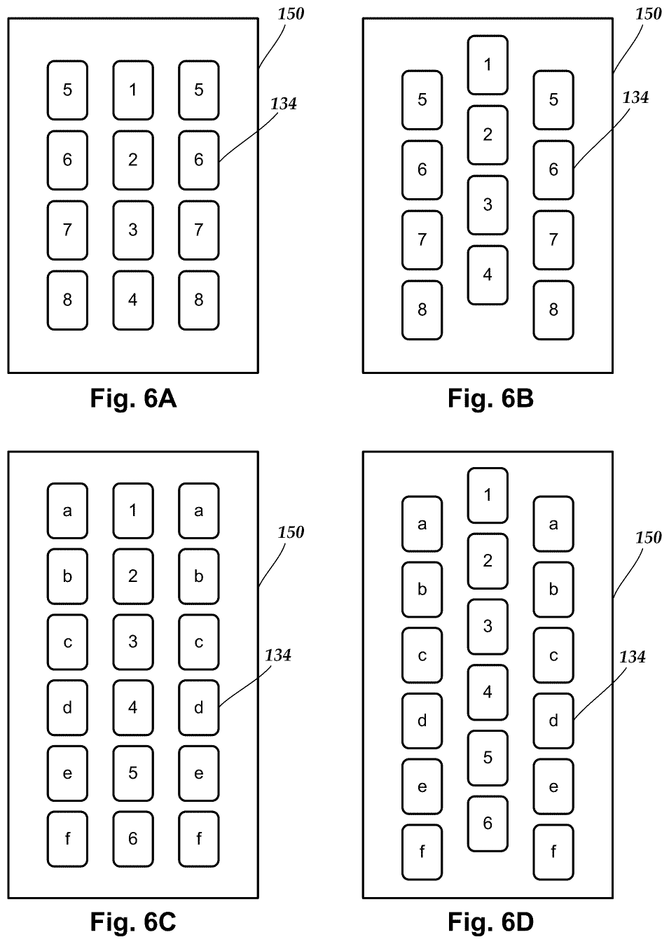

FIGS. 6A to 6D illustrate electrode arrangements in which corresponding electrodes in two rows are electrically coupled together (i.e., ganged together). In at least some embodiments, the housing or case of the control module can also act as a supplemental cathode or anode. In at least some embodiments, using the housing or case as a supplemental cathode or anode, can result in differentiation between the anodic amplitude and cathodic amplitude at the cuff. Such differentiation can produce or increase anodic (or cathodic) intensification or anodic (or cathodic) de-intensification.

FIGS. 6A and 6B illustrate three rows with four electrodes in each row (a 4-4-4 electrode arrangement) and FIGS. 6C and 6D illustrate three rows with six electrodes in each row (a 6-6-6 arrangement). The embodiments in FIGS. 6A and 6B use eight channels of an electrical stimulation system, as indicated by the numbering of the electrodes, and FIGS. 6C and 6D use 12 channels of an electrical stimulation system, although in both cases the number of electrodes exceeds the number of channels. In FIGS. 6B and 6D, the center row is staggered or offset relative to the two flanking rows. The corresponding electrodes in the outer two rows are electrically coupled together. For example, the two electrodes labeled “5” in FIGS. 6A and 6B are electrically coupled together. Similarly, the two electrodes labeled “6” are electrically coupled together, the two electrodes labeled “7” are electrically coupled together, and the two electrodes labeled “8” are electrically coupled together. For the twelve electrode embodiments in FIGS. 6C and 6D, the two electrodes labeled “a”, “b”, “c”, “d”, “e”, or “f”, respectively, are electrically coupled together.

Electrical coupling of two (or more) electrodes can be accomplished by, for example, coupling each of the two (or more) electrodes to the same conductor 160 in the lead body 106. One or more of the pairs of electrodes, acting as an anode, can provide anode intensification of the cathodic current through one of the electrodes of the middle row (for example, the electrode (e.g., electrode “1”) between the pair of electrodes (e.g., electrodes “5”)). Similarly, cathodic intensification by one or more of the pairs of the electrodes, acting as a cathode, of anodic current through one of the electrodes of the middle row can also be achieved. The intensification occurs as the current from the electrode of the middle row is directed much more in the vicinity of that electrode by the electrodes of opposite polarity that flank it.

In at least some embodiments, stimulation is provided by selecting one or more of the electrically coupled electrode pairs as one polarity (anodes) and the electrode between each of the electrode pairs as the other polarity (e.g., cathode(s)). Such arrangements can provide, for example, anode (or cathode) intensification using fewer stimulation channels.

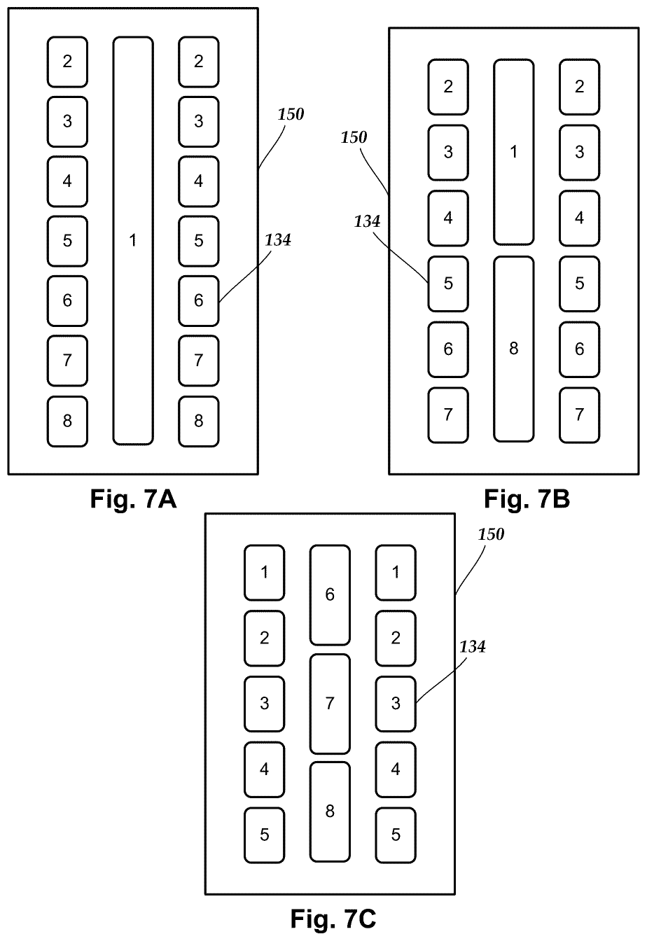

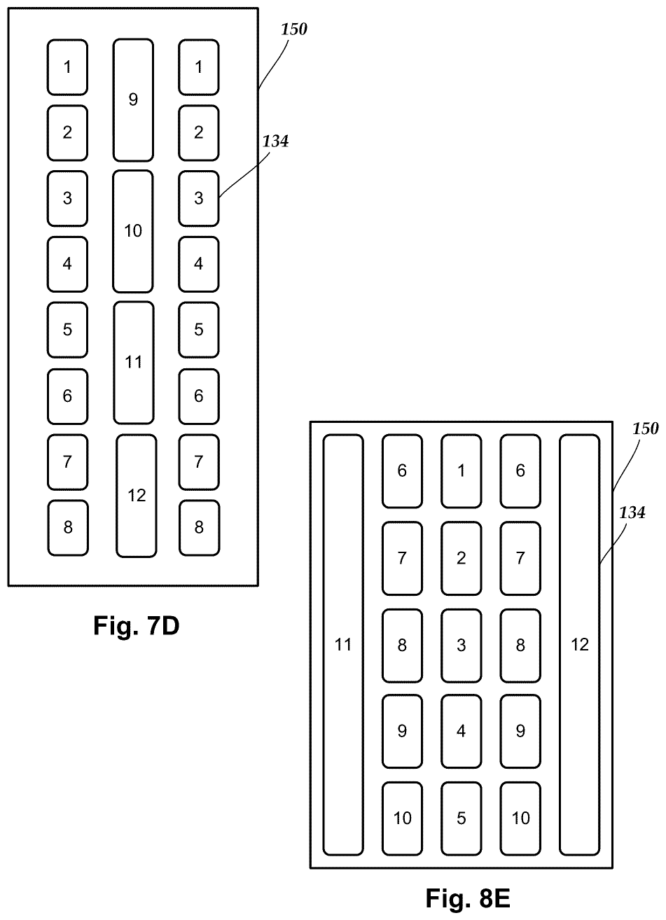

FIGS. 7A to 7D illustrate additional electrode arrangements that include one or more electrodes that are flanked by two rows of electrically coupled electrodes. The electrically coupled electrodes are shorter than the other electrodes. The electrode arrangement of FIG. 7A is a 7-1-7 arrangement with the electrodes of the first and third rows electrically coupled (i.e., ganged) together. The electrode arrangement of FIG. 7B is a 6-2-6 arrangement with the electrodes of the first and third rows electrically coupled (i.e., ganged) together. For example, the electrode arrangement of FIG. 7C is a 5-3-5 arrangement with the electrodes of the first and third rows electrically coupled (i.e., ganged) together. Other eight electrode arrangements include 3-5-3, 2-6-2, and 1-7-1. Different electrode arrangements can provide different amounts of electric field control, such as, for example, using the anode(s) to provide radial steering.

Similar arrangements can be provided for 12 electrodes. For example, the electrode arrangement of FIG. 7D is a 8-4-8 arrangement with the electrodes of the first and third rows electrically coupled (i.e., ganged) together. Similarly, arrangements having twelve electrodes with the first and third rows electrically coupled (i.e., ganged) together can include 11-1-11, 10-2-10, 9-3-9, 7-5-7, 6-6-6, 5-7-5, 4-8-4, 3-9-3, 2-10-2, and 1-11-1. A general arrangement is n-(N-n)-n where N is the total number of electrodes and n is the number of electrodes in each of the two rows that are electrically coupled together.

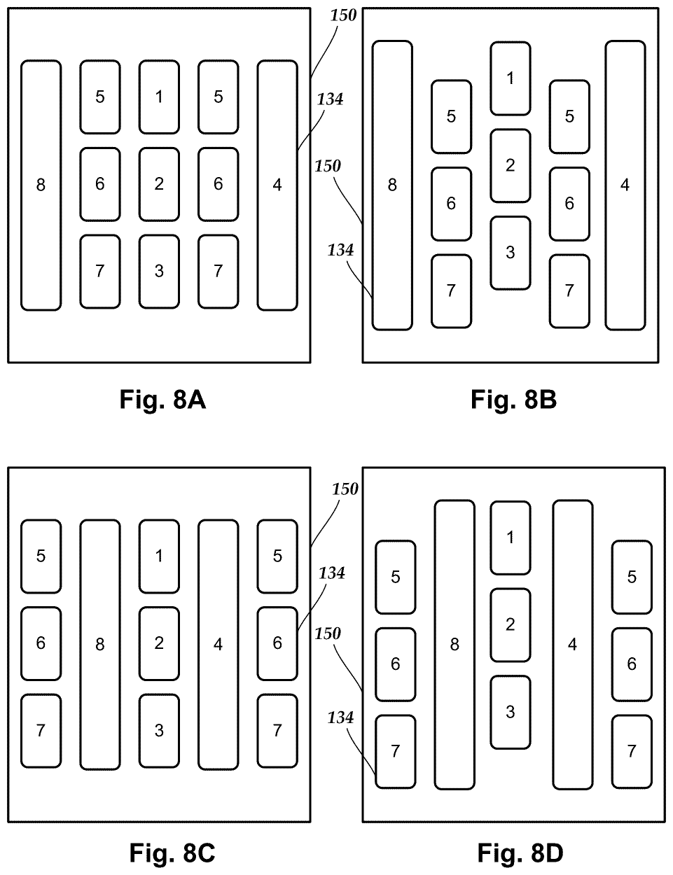

Arrangements with two rows of electrically coupled electrodes can also include additional rows of electrodes. FIGS. 8A to 8E illustrate electrode arrangements that include electrodes that are electrically coupled together (“5”, “6”, and “7” in FIGS. 8A to 8D and “6”, “7”, “8”, “9”, and “10” in FIG. 8E). These arrangement include single long electrodes (“4” and “8” in FIGS. 8A to 8D and “11” and “12” in FIG. 8E) that extend the length of a row and a set of other electrodes (including “1”, “2”, and “3” in FIGS. 8A to 8D and “1”, “2”, “3”, “4”, and “5” in FIG. 8E) that are disposed between the rows of the electrically coupled electrodes.

The electrode arrangements of FIGS. 8A and 8B are 1-3-3-3-1 arrangements with the electrodes of the second and fourth rows electrically coupled (e.g., ganged) together. In at least some embodiments, such an electrode arrangement can be used for tripolar stimulation. The electrode arrangements of FIGS. 8C and 8D are 3-1-3-1-3 arrangements with the electrodes of the first and fifth rows electrically coupled (e.g., ganged) together. The electrode arrangement of FIG. 8E is a 1-5-5-5-1 arrangement with the electrodes of the second and fourth rows electrically coupled (e.g., ganged) together. A twelve-electrode arrangement 5-1-5-1-5 is similar to the arrangements of FIGS. 8A and 8D. Examples of general arrangements for any number of electrodes include 1-n-(N-n-2)-n-1 or n-1-(N-n-2)-1-n, where N is the total number of electrodes and n is the number of electrodes in each of the two rows that are electrically coupled together.

As a modification to the arrangements of FIGS. 8A, 8B, and 8E, it will be understood that the two flanking rows can have more than one electrode and the electrodes of the second and fourth rows are electrically coupled in pairs, such the following electrode arrangements: 2-2-2-2-2, 2-3-5-3-2, 2-4-4-4-2, 3-3-3-3-3, or the like. As a modification to the arrangements of FIGS. 8C and 8D, it will be understood that the two flanking rows can have more than one electrode and the electrodes of the second and fourth rows are electrically coupled in pairs, such as the following electrode arrangements: 2-2-2-2-2, 2-3-5-3-2, 2-4-4-4-2, 3-3-3-3-3, or the like. Examples of general arrangements for any number of electrodes include m-n-(N-n-2m)-n-m or n-m-(N-n-2m)-m-n, where N is the total number of electrodes, n is the number of electrodes in each of the two rows that are electrically coupled together, and m is the number of electrode in two of the other rows.

FIGS. 4A, 4C, 5B, 5C, 5F, 7A, 7B, 7C, 7D, 8C, and 8D illustrate arrangements in which one or more rows (“type-L” rows) that contain one or more longer electrodes are disposed between rows (“type-S” rows) that contain shorter electrodes, where the longer electrodes have a longer length in the circumferential direction than the shorter electrodes. In at least some embodiments, the length in the circumferential direction of the longer electrodes is at least 1.5, 2, 2,5, 3, 4, or more times the length in the circumferential direction of the shorter electrodes. The

Any suitable methods can be used for manufacturing these cuffs. For example, conductors can be welded to the electrodes which are dispersed in a non-conducting (for example, silicone) carrier. A non-conductive layer can cover the conductors and this construct can be formed in the shape of a cuff. The cuff body can be formed of any suitable biocompatible and biostable non-conductive material including, but not limited to, polymer materials such as silicone, polyurethane, polyetheretherketone (“PEEK”), epoxy, or the like. Other techniques can be used to form the electrodes including, but not limited to, photolithography, e-beam lithography, electrodeposition, or sputtering of the electrodes on a substrate or laser cutting or ablation of electrode material disposed on/in, or disposable on/in, a non-conductive carrier.

In at least some embodiments, the electrodes are rectangular or rectangular with rounded corners. Any other suitable shape can be used for the electrodes including, but not limited to, oblong, oval, modified rectangular with one or more sides (or portions of sides) that are curved, or the like or any combination thereof. The electrodes can be formed using any conductive, biocompatible material. Examples of suitable materials include metals, alloys, conductive polymers, conductive carbon, and the like, as well as combinations thereof. In at least some embodiments, one or more of the electrodes are formed from one or more of: platinum, platinum alloys such as platinum iridium, palladium alloys such as palladium rhodium, titanium, titanium alloys, nickel alloys, cobalt alloys, nickel/cobalt alloys, stainless steels, tantalum, conductive carbon, conductive plastics, epoxy, or other adhesive filled with metallic powder, Nitinol™, or the like or any combination thereof.

In at least some embodiments, the electrodes have a contact surface that is flush or slightly protruding (for example, no more than 200, 100, or 50 μm) from the cuff body which, at least in some circumstances, may reduce or eliminate physical pressure on the nerve. It will be recognized that the electrodes can be used to provide electrical stimulation or to sense electrical signals from tissue or any combination thereof.

In at least some embodiments, the cuff 150 has an inner diameter in a range of 0.5 to 5.5 mm or in a range of 1 to 3 mm. In at least some embodiments, the cuff 150 has a length of at least 5, 10, 20, 30, 40, or 50 mm or more. In at least some embodiments, the cuff 150 is configured to fit around a portion of the vagus, splanchnic, hapatic, hypogastric, hypoglossal, sciatic, or other nerves.

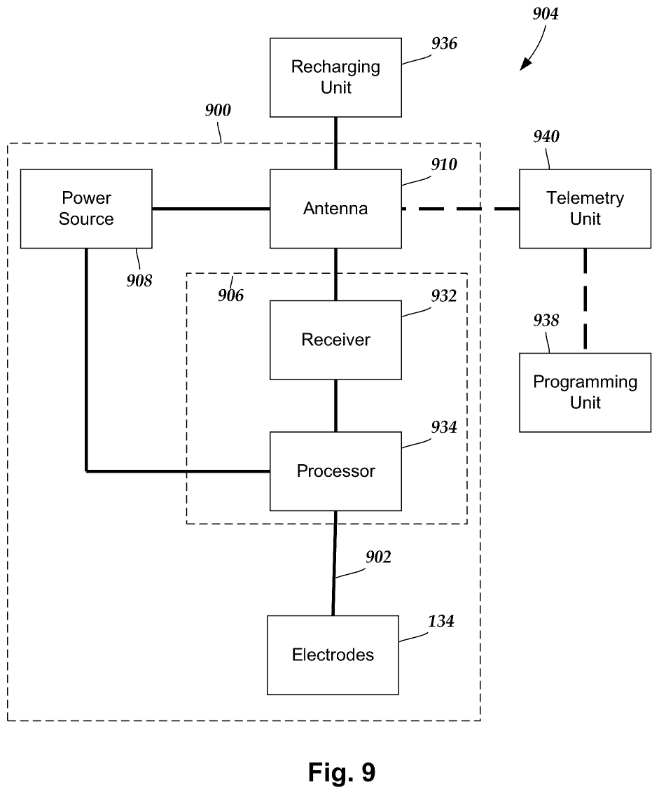

FIG. 9 is a schematic overview of one embodiment of components of an electrical stimulation arrangement 904 that includes an electrical stimulation system 900 with a lead 902, stimulation circuitry 906, a power source 908, and an antenna 910. The electrical stimulation system can be, for example, any of the electrical stimulation systems described above. It will be understood that the electrical stimulation arrangement can include more, fewer, or different components and can have a variety of different configurations including those configurations disclosed in the stimulator references cited herein.

If the power source 908 is a rechargeable battery or chargeable capacitor, the power source may be recharged/charged using the antenna 910, if desired. Power can be provided for recharging/charging by inductively coupling the power source 908 through the antenna 910 to a recharging unit 936 external to the user. Examples of such arrangements can be found in the references identified above.

In at least some embodiments, electrical current is emitted by the electrodes (such as electrodes 134 in FIG. 1) on the lead 902 to stimulate nerve fibers, muscle fibers, or other body tissues near the electrical stimulation system. The stimulation circuitry 906 can include, among other components, a processor 934 and a receiver 932. The processor 934 is included to control the timing and electrical characteristics of the electrical stimulation system. For example, the processor 934 can, if desired, control one or more of the timing, frequency, strength, duration, and waveform of the pulses. In addition, the processor 934 can select which electrodes can be used to provide stimulation, if desired (see, for example, U.S. Pat. No. 8,412,345, which is incorporated herein by reference in its entirety). In some embodiments, the processor 934 selects which electrode(s) are cathodes and which electrode(s) are anodes. In some embodiments, the processor 934 is used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 938 that, for example, allows modification of pulse characteristics. In the illustrated embodiment, the processor 934 is coupled to a receiver 932 which, in turn, is coupled to the antenna 910. This allows the processor 934 to receive instructions from an external source to, for example, direct the pulse characteristics and the selection of electrodes, if desired.

In at least some embodiments, the antenna 910 is capable of receiving signals (e.g., RF signals) from an external telemetry unit 940 that is programmed by the programming unit 938. The programming unit 938 can be external to, or part of, the telemetry unit 940. The telemetry unit 940 can be a device that is worn on the skin of the user or can be carried by the user and can have a form similar to a pager, cellular phone, or remote control, if desired. As another alternative, the telemetry unit 940 may not be worn or carried by the user but may only be available at a home station or at a clinician's office. The programming unit 938 can be any unit that can provide information to the telemetry unit 940 for transmission to the electrical stimulation system 900. The programming unit 938 can be part of the telemetry unit 940 or can provide signals or information to the telemetry unit 940 via a wireless or wired connection. One example of a suitable programming unit is a computer operated by the user or clinician to send signals to the telemetry unit 940.

The signals sent to the processor 934 via the antenna 910 and the receiver 932 can be used to modify or otherwise direct the operation of the electrical stimulation system 900. For example, the signals may be used to modify the pulses of the electrical stimulation system such as modifying one or more of pulse duration, pulse frequency, pulse waveform, and pulse strength. The signals may also direct the electrical stimulation system 900 to cease operation, to start operation, to start charging the battery, or to stop charging the battery.

Optionally, the electrical stimulation system 900 may include a transmitter (not shown) coupled to the processor 934 and the antenna 910 for transmitting signals back to the telemetry unit 940 or another unit capable of receiving the signals. For example, the electrical stimulation system 900 may transmit signals indicating whether the electrical stimulation system 900 is operating properly or not or indicating when the battery needs to be charged or the level of charge remaining in the battery. The processor 934 may also be capable of transmitting information about the pulse characteristics so that a user or clinician can determine or verify the characteristics.

The above specification provides a description of the structure, manufacture, and use of the invention. Since many embodiments of the invention can be made without departing from the spirit and scope of the invention, the invention also resides in the claims hereinafter appended.

What is claimed as new and desired to be protected is:

1. An electrical stimulation lead comprising:
a cuff comprising
a cuff body having an exterior surface and an interior surface, and
a plurality of electrodes disposed on the interior surface of the cuff body and arranged into at least three rows of electrodes, wherein the at least three rows comprises a first row, a second row, and a third row, wherein the second row is disposed between the first and third rows, wherein each electrode in the first row is electrically coupled to a corresponding electrode in the third row;
a lead body coupled to the cuff; and
a plurality of first conductors extending through the lead body and the cuff with the first conductors electrically coupled to the electrodes.

2. The electrical stimulation lead of claim 1, wherein each of the electrodes of the second row are staggered relative to the electrodes of the first and third rows.

3. The electrical stimulation lead of claim 1, wherein the first, second, and third rows have four electrodes each or six electrode each.

4. The electrical stimulation lead of claim 1, wherein the first and third rows have n electrodes each and the second row has N-n electrodes, wherein N is a total number of electrodes and n is less than N.

5. The electrical stimulation lead of claim 1, wherein the at least three rows further comprise a fourth row and a fifth row, wherein the first, second, and third rows are all disposed between fourth and fifth rows.

6. The electrical stimulation lead of claim 5, wherein the first and third rows have n electrodes each, the fourth and fifth rows have m electrodes each, and the second row has N-n-2m electrodes, wherein N is a total number of electrodes, n is less than N, m is less than N, and n-2m is less than N.

7. The electrical stimulation lead of claim 1, wherein the cuff further comprises a plurality of second conductors, wherein each of the second conductors electrically couples one of the electrodes of the first row to the corresponding one of the electrodes of the third row.

8. An electrical stimulation system, comprising:
the electrical stimulation lead of claim 1; and
a control module configured to receive a portion of the lead body of the electrical stimulation lead and to electrically couple to the electrodes of the cuff.

9. A method of electrically stimulating a patient, the method comprising:
providing the electrical stimulation lead of claim 1 implanted within the patient; and
applying electrical current through the patient with at least one of the electrodes of each of the first and third rows having a first polarity and at least one of the electrodes of the second row having a second polarity opposite the first polarity.

10. An electrical stimulation lead comprising:
a cuff comprising
a cuff body having an exterior surface and an interior surface, and
a plurality of electrodes disposed on the interior surface of the cuff body and arranged into at least three rows of electrodes, wherein the at least three rows comprises a first row, a second row, and a third row, wherein the second row is disposed between the first and third rows, wherein the first and third rows each comprise a plurality of the electrodes and the second row comprises at least one electrode, wherein each of the at least one electrode in the second row is at least twice as long as each of the electrodes of the first and third rows;
a lead body coupled to the cuff; and
a plurality of conductors extending through the lead body and the cuff with the conductors electrically coupled to the electrodes.

11. The electrical stimulation lead of claim 10, wherein the at least three rows further comprises a fourth row disposed between the first and third rows, wherein the fourth row comprises at least one electrode, wherein each of the at least one electrode of the fourth row is at least twice as long as each of the electrodes of the first and third rows.

12. The electrical stimulation lead of claim 11, wherein the at least three rows further comprises a fifth row that is either a) not disposed between the first and third rows, wherein the fifth row comprises at least one electrode, wherein each of the at least one electrode of the fifth row is at least twice as long as each of the electrodes of the first and third rows or b) between the second and fourth rows, wherein the fifth row comprises at least one electrode.

13. The electrical stimulation lead of claim 10, wherein the at least three rows further comprises a fourth row that is not disposed between the first and third rows, wherein the fourth row comprises at least one electrode, wherein each of the at least one electrode of the fourth row is at least twice as long as each of the electrodes of the first and third rows.

14. The electrical stimulation lead of claim 10, wherein each of the electrodes of the first row is electrically coupled to a corresponding one of the electrodes of the third row.

15. The electrical stimulation lead of claim 10, wherein the second row comprises a) a plurality of the electrode or b) a single one of the electrodes that is at least as long as a combined length of all of the electrodes of the first row.

16. An electrical stimulation lead comprising:
a cuff comprising
a cuff body having an exterior surface and an interior surface, and
up to sixteen electrodes disposed on the interior surface of the cuff body and arranged into at least three rows of electrodes, wherein the at least three rows comprises a first row, a second row, and a third row, wherein the second row is disposed between the first and third rows and comprises more of the electrodes than the first row and more of the electrodes than the third row, wherein each electrode in the first and third rows is at least twice as long as each of the electrodes of the second row;
a lead body coupled to the cuff; and
a plurality of conductors extending through the lead body and the cuff with the conductors electrically coupled to the electrodes.

17. The electrical stimulation lead of claim 16, wherein the first and third electrodes each comprise a plurality of the electrodes.

18. The electrical stimulation lead of claim 16, wherein the at least three rows further comprises a fourth row that is not disposed between the first and third rows.

19. The electrical stimulation lead of claim 16, wherein the at least three rows further comprises a fourth row that is disposed between the first and third rows.

20. The electrical stimulation lead of claim 16, wherein each of the electrodes of the first row is electrically coupled to a corresponding one of the electrodes of the third row.
